(19) United States
(12) Patent Application Publication
(10) Pub. No.: US 2008/0057114 A1
(43) Pub. Date: Mar. 6, 2008

(54) Sinker capsules

(76) Inventor: Gregory Dale Matthews, El Paso, TX (US)

Correspondence Address: GREGOR DALE MATTHEWS, 1309 YVONNE DIANE DR., EL PASO, TX, 79936, US

(21) Appl. No.: 11/513,617

(22) Filed: Sep. 1, 2006

(51) Int. Cl. A61K 9/48

(52) U.S. Cl. 424/451; 424/776

(57) ABSTRACT

Sinker Capsules are gelatin capsules with two halves that are filled with a dense material, such as chocolate or other similiar material, in one half and medication in the other half. The bottonm part of the capsule is larger and is filled with medication. The medication is pressed into the lower half of the capsule. The smaller, upper part of the capsule is filled with a dense material for the purpose of adding weight to the capsule. When the two halves of the capsule are joined together, they form a safe delivery system for the patient's medication. It is then swallowed with a cool glass of water on an empty stomach. The Sinker Capsule will then sink to the bottom of the stomach. It then enters the intestines without dissolving in the stomach. This enables the patient to take their medication without irritating the lining of the stomach.

### FIELD OF THE INVENTION

The present invention relates to capsules and, more particularly, to a capsule in which different medications can be placed and which upon swallowing will sink immediately to the bottom of the stomach.

### BACKGROUND OF THE INVENTION

Every since people have been taking medications by mouth, there has been a need to protect the stomach from harmful irritation. Medications that are taken daily for chronic diseases can cause irritation to the lining of the stomach. This can lead to gastritis and ulcers. Some solutions have been to take medications with food. Also doctors will prescribe additional medications such as Antacids and anti ulcer medications like Pepcid to protect the stomach. Some tablets have coatings on them to prevent them from dissolving in the stomach, The problem is that not all medications have the proper coating to safely by pass the stomach. Sinker Capsules are designed to be versatile. A patient can use these capsules to be able to swallow ANY medication without hurting the stomach. Some medications, such as arthritis medications are usually irritating to the stomach. These can be incorporated into Sinker Capsules and will by pass the stomach without causing any injury to the stomach.

Other solutions such as coating tablets are used only for a specific commercially available product.

Some medications have been coated to protect the stomach. Others are designed to dissolve slowly or to dissolve only in the intestines. But these applications are for specific products. The problem is that there does not exist a versatile or adaptable capsule that can be used on any medication. For example, all forms of prednisone tablets are irritating to the stomach. There are no commercially available products that are safely coated. If a person has an active ulcer, his only option would be to inject the medication. Sinker Capsules enables that person to be able to take prednisone by mouth daily without hurting the stomach. For that matther Sinker Capsules can enable a person to take any medication by mouth without hurting the stomach.

It is therefore an object of the invention to enable persons with sensitive stomach to take any medication they need.

It is another object of the invention to enable people with chronic diseases such as arthritis to be able to take anti-inflamatory medications daily without hurting their stomach.

It is another object of the invention to give patients with ulcers more choices as to which medication they can take for their health problem . . .

It is another object of the invention to prevent patients from ever getting ulcers by taken their medication in a safer delivery system.

### SUMMARY OF THE INVENTION

In accordance with the present invention, there is provided a capsule consisting of two parts. The bottonm part of the capsule is larger and is filled with an exact amount of medication. The smaller upper part of the capsule is filled with a dense material for the purpose of adding weight to the capsule.

The dense materical, such as chocolate, or similiar material, is added in the top part of the capsule because in manufacturing Sinker Capsules this is the only area available.

When the two halves of the capsule are joined together, they form a safe delivery system for the patient's medication which will, when swallowed, immediately sink to the bottom of the stomach. It then enters the intestines without ever dissolving in the stomach. This enables the patient to take their medication without irritating the lining of the stomach or without causing ulcers.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a perspective view of a FIG. 1 shows a perspective view of a sinker capsule in accordance with the invention.

For purposes of clarity and brevity, like elements and components will bear the same designations and numbering throughout the Figures.

### DESCRIPTION OF THE PREFERRED EMBODIMENT

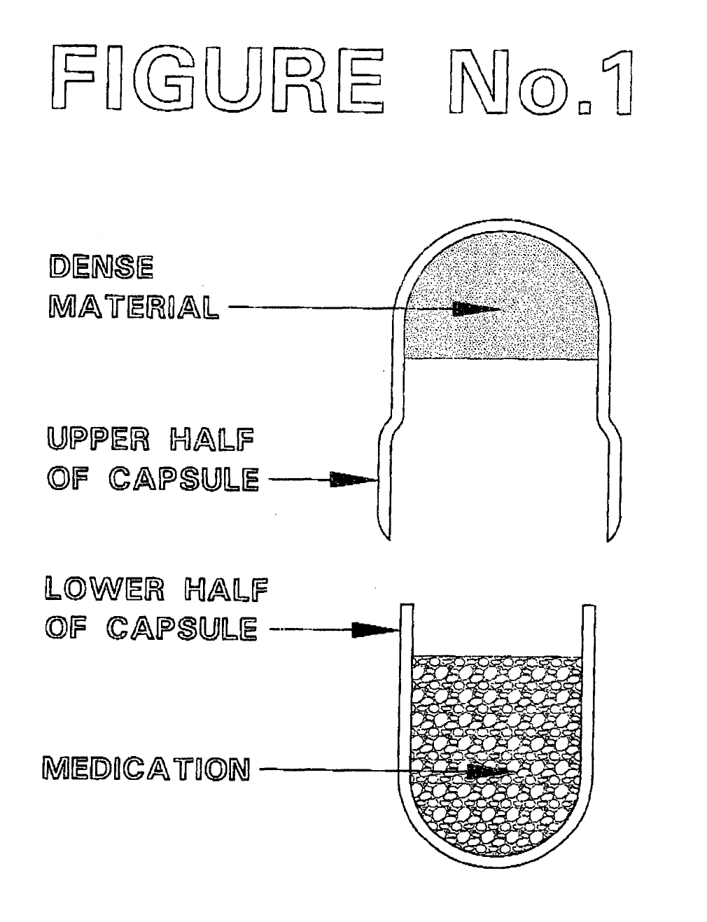

FIG. 1 is a perspective view of a Sinker Capsule in accordance with the invention. A pharmacist, acting in compliance with state law, and according to the authority of a written prescription from a physician, will load the lower half of the capsule with an exact amount of Medication 16. Then the lid of the capsule, the Upper half of the capsule 10, is loaded with a small amount of Dense material 12. This Dense material 12, being denser than water, will in effect cause the entire capsule to sink. The capsule is then closed securely by pressing firmly on it. When the capsule is taken on an empty stomach with a cool glass of water or milk, the Sinker Capsule will immediately sink to the bottom of the stomach without dissolving. It then enters the intestines were it will take several hours to dissolve. This eliminates any irritation from the Medication 16 to the lining of the stomach.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A sinker capsules for a sinker capsule for delivering medications into and through the stomach without causing irritation to the lining of the stomach, comprising:
means for holding the dense material, such as chocolate or similiar material;
means for causeing the entire capsule with its medication to sink immediately to the bottom of the stomach, securably embedded to said means for holding the dense material, such as chocolate or similiar material;
means for holding a specific amount of medication; and
means for treating the patient's disease, firmly embedded to said means for holding a specific amount of medication.

2. The sinker capsules in accordance with claim 1, wherein said means for holding the dense material, such as chocolate or similiar material comprises a round, hollow upper half of the capsule.

3. The sinker capsules in accordance with claim 1, wherein said means for causeing the entire capsule with its medication to sink immediately to the bottom of the stomach comprises a heavier than water dense material.

4. The sinker capsules in accordance with claim 1, wherein said means for holding a specific amount of medication comprises a round, hollow lower half of the capsule.

5. The sinker capsules in accordance with claim 1, wherein said means for treating the patient's disease comprises a dissolves slowly in intestines medication.

6. A sinker capsules for a sinker capsule for delivering medications into and through the stomach without causing irritation to the lining of the stomach, comprising:
a round, hollow upper half of the capsule, for holding the dense material, such as chocolate or similiar material;
a heavier than water dense material, for causeing the entire capsule with its medication to sink immediately to the bottom of the stomach, securably embedded to said upper half of the capsule;
a round, hollow lower half of the capsule, for holding a specific amount of medication; and
a dissolves slowly in intestines medication, for treating the patient's disease, firmly embedded to said lower half of the capsule

7. A sinker capsules for a sinker capsule for delivering medications into and through the stomach without causing irritation to the lining of the stomach, comprising:
a round, hollow upper half of the capsule, for holding the dense material, such as chocolate or similiar material;
a heavier than water dense material, for causeing the entire capsule with its medication to sink immediately to the bottom of the stomach, securably embedded to said upper half of the capsule;
a round, hollow lower half of the capsule, for holding a specific amount of medication; and
a dissolves slowly in intestines medication, for treating the patient's disease, firmly embedded to said lower half of the capsule
